(19) United States
(12) Patent Application Publication
(10) Pub. No.: US 2008/0039826 A1
(43) Pub. Date: Feb. 14, 2008

(54) Laser treatment

(76) Inventor: Adrianna Scheibner, Beverly Hills, CA (US)

Correspondence Address: HAHN LOESER & PARKS, LLP, One GOJO Plaza, Suite 300, AKRON, OH, 44311-1076, US

(21) Appl. No.: 11/549,465

(22) Filed: Oct. 13, 2006

(30) Foreign Application Priority Data
Apr. 13, 2004 (AU) 2004901906

(51) Int. Cl. A61N 5/067

(52) U.S. Cl. 606/9

(57) ABSTRACT

A method of laser dermatology comprising applying to skin laser energy suitable for dermatological treatment. The laser energy is applied to the skin having angle of incidence to the skin in the range of about 3° to 33°.

### CROSS-REFERENCE TO RELATED APPLICATIONS

This application is a continuation of Application No. PCT/IB2005/051208, filed Apr. 12, 2005, which is hereby incorporated herein by reference in its entirety.

### FIELD OF THE INVENTION

The present invention relates to the laser treatment of the body for the purpose of remedying various defects, problems and conditions. These defects may include skin conditions or problems (including purely cosmetic skin defects), conditions involving the gum or mucosa, scarring, allergies and internal bodily problems.

### BACKGROUND OF THE INVENTION

The use of various types of lasers for medical procedures was developed in the 1960's where lasers were first used as cutting tools in operations by some surgeons. Application of lasers for use in dermatology commenced in the early 1960's, but was generally limited to the treatment of tattoos and melanoma, and these experimental techniques were eventually abandoned as the rate of tissue scarring was unacceptably high. By the late 1970's, it had become possible to control laser energies to lower levels than those used originally, and as such successful treatment of tattoos was reported. However, the energy levels used were still so high that the associated pain usually required general anaesthesia.

Over the past few decades laser technology has developed to enable treatment of a variety of different skin conditions, ranging from treating the effects of sun damage to the skin through to the removal of body hair, and more recently the treatment of psoriases and acne.

Australian Patent Application No. AU-A-55004/94, filed in the name of the present Applicant, described one such method of utilising laser technology to treat a variety of skin conditions such as scars, pigmentation problems, wrinkles, psoriasis, adenoma, sebaceum, etc. in both adults and children. This method generally related to directing the laser light to impinge upon the affected tissue site at a considerably high intensity in the blue, green, yellow, or orange spectral wavelength range. Whilst this method has proven successful in treating a variety of conditions not previously possible with earlier laser treatments, the success of the method relied upon the considerable skill, care and attention of the operator to effectively control the considerably high intensity laser light.

More recently, lasers have been employed to perform skin resurfacing procedures in an attempt to minimise the appearance of lines and blemishes on the skin and rejuvenate the skin. In such procedures, the laser is utilised such that an intense burst of laser energy is directed on the surface of the skin, heating the water contained within the surface layers of the skin, such that both the water and the skin vaporises. By passing the high level intensity laser across the desired region of the skin in a precise and controlled manner, the epidermis and the superficial reticular dermis can be removed or “ablated”. Over time, the skin is able to heal as new layers of collagen are produced and there is a contraction of the skin due to sufficient but not excessive dermal collagen heating by the laser, which can reduce the appearance of lines, wrinkles, marks and the like, which may have been present on the skin. It will be appreciated however, that such a procedure destroys the surface of the skin thereby causing pain and discomfort due to burning/destruction of the epidermis and as such requires local or general anaesthesia. Furthermore, following the procedure, considerable swelling and discomfort and in some cases permanent scarring may be experienced.

There is therefore a need to provide a relatively simple method of utilising low-level intensity laser energy to provide controlled dermal collagen heating to treat a variety of skin conditions without requiring destruction of the epidermis and the related side effects associated therewith.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### SUMMARY OF THE INVENTION

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

According to a first aspect of the present invention, there is provided a method of laser dermatology comprising applying to skin a laser energy suitable for dermatological treatment whereby the laser energy is applied at an angle of incidence to the skin in the range of about 3° to 33°, more preferably 10° to 26°, and most preferably around 18°.

According to a second aspect of the present invention, there is provided a method of reducing skin disfigurements comprising applying to the disfigured skin a laser energy from a laser energy source, said laser energy source being suitable for dermatological treatment, wherein the laser energy is applied at an angle of incidence to the skin in the range of about 3° to 33°, more preferably 10° to 26°, and most preferably around 18°, and the laser energy source is in a non-contact arrangement with the skin, preferably at a distance between about 0.5 cm and 30 cm from the skin.

According to a third aspect of the present invention, there is provided a method of fusing two edges of skin at a surgical site comprising applying to skin a laser energy from a laser energy source, said laser energy source being suitable for dermatological treatment, wherein the laser energy is applied at an angle of incidence to the skin in the range of about 3° to 33°, more preferably 10° to 26°, and most preferably around 18°, and the laser energy source is in a non-contact arrangement with the skin, preferably arranged at a distance between about 0.5 cm and 30 cm from the skin.

The method according to the third aspect of the present invention may be carried out immediately following an invasive surgical procedure to fuse the opening of the surgical site to prevent or reduce scarring at the surgical site. The cut edges of the scar are melted and fused together to facilitate rapid healing at the surgical site and reduce pain associated with the surgery. Further, following this application of this method, the skin at the surgical site is inhibited from stretching after surgery, and the likelihood of scarring after surgery is reduced or eliminated. Similarly, the method according to the third aspect may be carried out upon an open wound, such as an ulcer or the like, to facilitate healing and closure of the wound.

According to a fourth aspect of the present invention, there is provided a method of reducing the appearance of irregular skin colourations comprising the steps of:

puncturing the skin with relatively dry needles; and

applying to the skin a laser energy from a laser energy source, the laser energy source being suitable for dermatological treatment, wherein the laser energy is applied at an angle of incidence to the skin in the range of about 3° to 33°, more preferably 10° to 26°, and most preferably around 18°, and the laser energy source is in a non-contact arrangement with the skin, preferably arranged at a distance of between about 0.5 cm and about 30 cm from the skin.

Following treatment with the laser energy in the method according to the fourth aspect of the present invention, the area of laser-treated skin may be topically applied with comfrey and/or a moisturising cream to facilitate rapid healing.

By “irregular skin colourations” is meant any unusual or abnormal colouration of the skin. Examples of irregular colouring of the skin include conditions caused by vitiligo, white scars or tattooing. This method provides a safe and effective method for reducing the appearance of irregular skin colourations caused by skin conditions such as vitiligo and white scars, or caused by tattooing.

Preferably, the method according to any one of the first to the fourth aspects of the present invention is conducted to confine the temperature of the dermis portion of skin under treatment to a temperature increase to a value not above 80° C. and preferably the method achieves an increase of dermis skin temperature to a value in the range 50° to 80° C.

The method according to any one of the first to the fourth aspects of the present invention may be applied by using a guide for the laser beam so that resting the guide itself or the hand of the operator on the surface of the skin maintains the desired angle of incidence. The guide used may be and preferably is one of the preferred versions described below.

Preferably, the method according to any one of the first to the fourth aspects of the present invention includes using a thin small zone of laser energy applied to the skin at an acute angle of incidence, the size of the spot depending on the angle of incidence. Generally, the invention may be applied using a laser emission with diameter 1 to 10 mm and preferably around 5 mm.

In the method according to any one of the first to the fourth aspects of the present invention, it has been found that by controlling movement of the laser in a manner which correlates with the energy of the beam, the preferred elevation of dermis skin temperature is achieved and careful movement of the spot of laser light can permit a substantial area to be treated in a session.

According to a fifth aspect of the present invention, there is provided a method of stimulating hair growth or gum/mucosa regeneration in an area of skin or gum/mucosa where hair or gum/mucosa growth has receded or ceased or become sparse, comprising applying to the skin or gum/mucosa a laser energy from a laser energy source, said laser energy source being suitable for medical use, wherein the laser energy is applied at an angle of incidence to the skin or gum/mucosa in the range of about 25° to 65°, most preferably about 45°, and the laser energy source is in a non-contact arrangement with the skin or gum/mucosa, preferably arranged at a distance of between about 0.5 cm and about 30 cm from the skin or gum/mucosa.

Hair loss arising from balding or stress may be treated using the method according to the fifth aspect of the present invention. Recession of gum or mucosa growth arising from stress or disease may also be treated using the method according to the fifth aspect of the present invention.

According to a sixth aspect of the present invention, there is provided a method of treating acne comprising applying to the skin a laser energy, suitable for dermatological treatment, whereby the laser energy is applied at an angle of incidence to the skin in the range of about 25° to 65°, most preferably about 45°, and from a distance between about 0.5 cm and about 30 cm from the skin.

According to a seventh aspect of the present invention, there is provided a method of laser-accelerated healing of a patient in need thereof comprising applying to patient a laser energy, suitable for medical treatment, wherein the laser energy is applied to the patient from a distance of between about 30 cm and 5 m.

It has been found that the method according to the seventh aspect of the present invention, which can utilise conventional lasers manufactured for medical use, has a significant and beneficial effect in treating many different conditions including burns, recent and chronic injuries, arthritis, stiff joints, skin conditions, active acne, psoriasis, dermatitis, warts, viral infections, allergies, asthma, and internal bodily problems. The method is also effective in assisting in pain management and wound healing. This method is also useful in treating animals with similar ailments and conditions. Surprisingly, this method also may enhance the growth of plants.

Advantageously, the method according to the seventh aspect of the present invention may be used on a patient in conjunction with the method according to any one of the first to the sixth aspects of the invention. The method according to the seventh aspect of the present invention may be carried out either before or after carrying out a method according to any one of the first to the sixth aspects of the present invention to enhance the results of the method according to any one of the first to the sixth aspect of the present invention.

Preferably, the method according to any one of the fifth to the seventh aspects of the present invention is conducted to confine the portion of skin under treatment to a temperature increase to a value between 0° C. to about 30° C. Typically, there is a temperature increase in the portion of skin under treatment with the method according to the sixth aspect of the present invention of about 5 to 30° C.

The method according to any one of the fifth to the seventh aspects of the present invention may be applied to the patient by waving the laser energy at the patient from a distance between about 30 cm to 5 m from the patient.

Preferably, the method according to any one of the fifth to the seventh aspects of the present invention includes using a wide zone of laser energy applied to the skin at varying angles of incidence, the size of the spot depending on the angle of incidence. Generally, the invention may be applied using a laser emission with diameter 1 mm to 100 mm, but the size of the beam is not crucial to the working of the method.

Further, the angle of incidence in the method according to any one of the fifth to the seventh aspects of the present invention is much greater than in the method according to any one of the first to the fourth aspects of the present invention as the laser energy is applied to the patient from a distance between about 30 cm and about 5 m from the patient. The precision of the laser energy is not important, so long as there is exposure of the patient to the laser energy.

Surprisingly, in the method according to any one of the fifth to the seventh aspects of the present invention, it has been found there is little or no obvious immediate effect of the treatment although some slight redness reaction may be apparent to the patient for a few hours or a few days.

A particularly effective laser for use in each of the aspects of the present invention is a low energy CO₂ laser but other medically suitable substitutes could be used. Preferably, the laser light-utilised for the first to the seventh aspects of the present invention is a low energy CO₂ laser operated at a wavelength of about 10,600 nm operated at between about 0.5 and about 5 watts. However, other medically suitable lasers may be used to carry out the present invention. For example, any medically suitable laser operating at a wavelength in the range of about 400 to about 10,600 nm may be used, including an argon laser, an argon dye laser, a diode laser, a KTP laser, a copper vapour laser, a Yag laser, a ruby laser and a flashlamp-pumped dye laser. Further, an IPL (intense pulsed light) “laser” (though technically not a laser) may be used to achieve the results of the methods of the present invention. It is intended that the IPL (intense pulsed light) “laser” is included within the scope of lasers used in the present invention.

A series of treatment procedures of the method according to any one of the first to the seventh aspects of the present invention spaced several months apart may be required but even a single treatment can be highly beneficial at remedying the problem.

According to an eighth aspect of the invention, there is provided a device for laser treatment of a patient comprising

a laser energy source for generating a beam of laser energy; and

a guide for receiving said laser energy source and for supporting said laser energy source above, but not in contact with, the skin of the patient, said guide having a laser energy discharge port through which said beam of laser energy can pass from said laser energy source to the skin of the patient, said guide being positionable such that the device can be moved over and adjacent to the skin of the patient while maintaining an angle of incidence of the discharged laser energy to the skin in a range of about 3° to about 65°, e.g. 3° to 33° or 25° to 65°.

Different forms of device can be employed. One type of device can include a mirror or other optical device whereby a steeper angle of incidence from the laser is provided in order to reflect the laser beam to the desired angle of incidence. In this form the device could be adapted to be held in the manner of a pen at a comfortable angle of incidence of around 45° (say 25° to 65°) with a mirror set up to provide the required reflection. In this embodiment the tip of the device can be curved and adapted to be passed over and touch the skin of the patient with a discharge aperture spaced from the tip to project the laser light to a location spaced from the tip. As such, the tip of the device acts as a supporting and guiding element for the laser, thereby preventing direct contact of the laser with the skin or surrounding tissue.

Another form of device is one having an elongate hand and finger grip adapted to be held like a pen and to have a tip which may be passed over the skin of the patient and a laser light application guide suspended below the hand grip for accommodating the output from a laser and directing the light at the required angle of incidence onto the skin at a location adjacent but typically behind the location at which the tip of the handgrip is adapted to touch the skin.

A further form of device is one which can be handheld or mounted on a platform for use in the seventh aspect of the present invention. The device may be waved by the user's hand so as to direct the laser light onto the patient from above or from the side of the patient. Alternatively, the device can be mounted on a platform so as to direct the laser light on the patient from above or from the side of the patient. The patient may be standing, seated or lying down when being treated with the laser.

### BRIEF DESCRIPTION OF THE DRAWINGS

By way of example only, preferred embodiments of the invention are now described with reference to the accompanying drawings, in which:

FIG. 1 is an overall side view of a first embodiment of the invention;

FIG. 2 is an enlarged view of FIG. 1 illustrating application of the laser beam;

FIG. 3 is an overall side view of a second embodiment of a device for using the present invention;

FIG. 4 is an enlarged view of the device of FIG. 3 illustrating the optical path;

FIG. 5A is a photographic reproduction of an elderly patient before treatment to a face region;

FIG. 5B is a photograph of the patient of FIG. 5A after a recovery period has occurred from treatment in accordance with the invention;

FIG. 6A is a photographic reproduction of an elderly patient before treatment to an eye region;

FIG. 6B is a photograph of the patient of FIG. 6A after a recovery period has occurred from treatment in accordance with the invention; and

FIGS. 7A-7C is a photographic reproduction of an ulcer on the sole of a patient's foot, before treatment, six weeks after a first treatment, and two months after a second treatment respectively, in accordance with the invention.

### PREFERRED MODE OF CARRYING OUT THE INVENTION

The human skin can generally be considered to consist of three layers, the epidermis, the dermis, and the hypodermis.

The epidermis is the top layer of the skin and largely acts as a barrier to the external environment to seal and protect the body from infection and to retain moisture within the body. The epidermis is generally built up of several layers of living cells and top layers of dead cells, and is constantly growing with new cells being created at its base and old cells quickly dying. In this regard, as old skin cells die, they are pushed toward the surface by the constantly growing new cells where the dead cells eventually flake away. The epidermis is also responsible for the colouring of the skin as it holds the skins pigment and its thickness can vary significantly, ranging from between 0.05 mm and 1.5 mm.

Below the epidermis is the dermis which essentially contains the structural elements of the skin, which is composed entirely of living cells and can be up to 3000 microns in thickness. The dermis contains: collagen, a protein which gives the skin its strength; glycosaminoglycans, proteins which give the skin its turgor; and elastin fibers, proteins which give the skin its elasticity or spring. As the epidermis does not have its own direct blood supply, the dermis supplies all nutrients to feed the cells of the epidermis and determines the tone of the skin.

The hypodermis lies beneath the dermis and contains the bases of the hair follicles and sweat glands and is the thickest layer of the skin. The hypodermis also contains cells capable of accumulating and storing fats which aid in insulating the body and acting as an energy reserve for the body. The hypodermis also provides heat conservation and acts as a cushion for the rest of the skin.

As discussed above, the collagen and elastin fibres present in the dermis are responsible for the resilience and elasticity of the skin. Therefore, if the skin becomes damaged as a result of old age or due to a disease or skin condition or exposure to sun and the like, the ability of the skin to produce elastin and collagen is affected, and hence the condition of the skin changes.

As discussed previously, one method of treating damaged skin to regenerate the skin's natural production of collagen and elastin, has been to undergo a “skin resurfacing” treatment. In this regard, the epidermis is typically removed in a precise and controlled manner to an appropriate depth by laser treatment, mechanical abrasion or by a chemical removal process. Following removal of the layers of skin, the skin is allowed to heal, thereby enabling new layers of collagen to be produced. Hence the skin essentially resurfaces itself to reduce the appearance of lines, wrinkles, scars etc which may be present on the surface of the skin. However in many cases, these methods have the potential to cause further damage to the already damaged skin and as it requires destroying/removing layers of skin, the treatment is painful and requires much healing time. Hence such methods of regenerating the skins natural production of collagen and elastin have been found to have many drawbacks.

As collagen and the other proteins are present in the dermis and account for up to 75% of the weight of the dermis, the present invention is directed towards treating this layer of the skin directly, rather than destroying the epidermis layers of the skin to access the dermis. In this regard, it has been found that by heating the dermis layer to a temperature of between 50°-80° C., the proteins present in the dermis layer are stimulated thereby triggering skin rejuvenation without tissue loss.

Heating of the dermis layer to these temperatures without damaging the epidermis is achievable by applying a low level laser light at an appropriate wavelength to the affected area of the skin to achieve the appropriate depth of penetration of the laser energy. By controlling the angle of incidence of the laser light to the skin, it has been found that due to internal reflection issues related to the beam of laser energy, the laser energy is more readily contained within the dermis layer and able to more effectively heat the dermis layer and the associated collagen retained therein, to trigger the desired rejuvenation effect.

It has been found that an angle of incidence of 18° is ideal to assist in concentrating and retaining the beam 16 of laser light at the desired skin depth to ensure that appropriate heating of the dermis layer is achieved to stimulate the collagen. However other angles of incidence are also possible and can range anywhere between 3° to 33°.

The affected area of skin may be an area of skin affected by scar tissue resulting from an accident or surgery, or may be an area of skin affected by stretchmarks, white scars, broken capillaries or sun damage. The affected area may also be an open wound, such as an ulcer or the like, whereby the edges of the skin are not fused together. Similarly, the affected area of the skin may be an area of the skin having irregular skin colourations, which may be due to a variety of conditions, such as vitiligo, white scars or tattooing. Therefore by concentrating laser light at the dermis of the affected area and raising the temperature of this region to between 50°-80° C., more preferably 50°-65° C., regeneration of healthy skin tissue can be triggered and the previously subsisting defective skin structure or unattractive appearance can be overcome as a result.

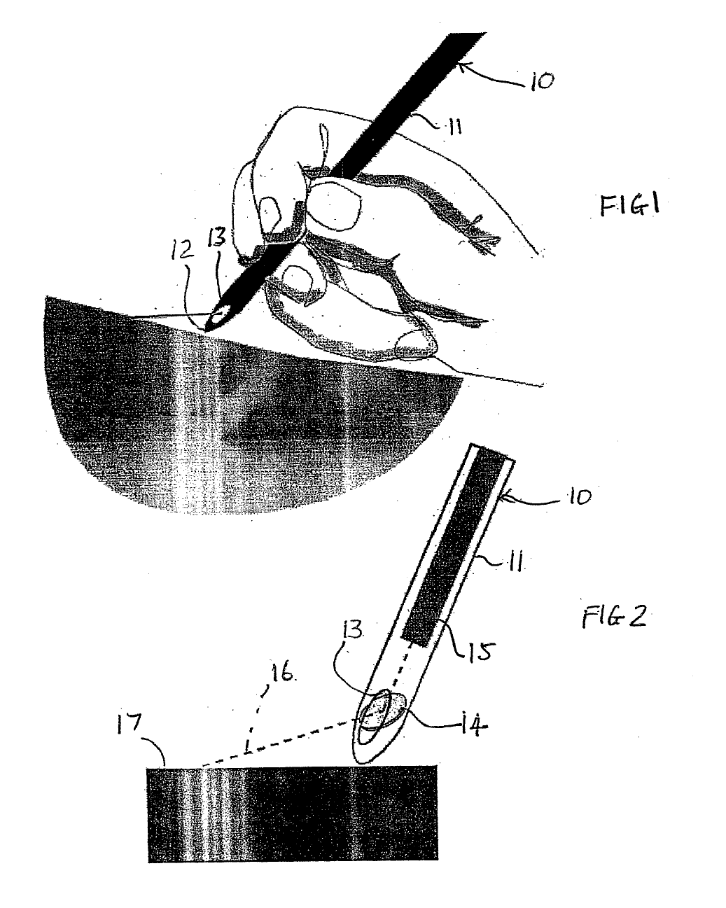

With reference to FIG. 1, a laser applicator tool 10 suitable for performing this procedure is shown. The tool 10, generally comprises an elongate barrel 11 having a smooth tip 12 and an elliptical opening 13 near the tip. As is shown in FIG. 2, a mirror 14 is accommodated within the elliptical opening of the tool 10 to reflect laser light from a laser optical discharge unit 15 to form a beam 16 which becomes incident on the patient's skin 17 at a desired angle of approximately 18°. The device permits the operator to conveniently hold the tool 10 in the hand as illustrated in FIG. 1 and to progressively move the device over the skin 17 for treatment purposes thereby retaining the desired angle of incidence of the beam 16 of laser light on the patient's skin 17. By resting the tool 10 or the hand of the operator on the surface of the skin during the procedure, the desired angle of incidence of the beam 16 is maintained, as is the distance at which the laser light is applied to ensure optimum results.

It has also been found that the distance at which the laser light is applied to the surface of the skin is important in achieving desired heating of the dermis. In this regard, it has been found that a distance of between 0.5 cm and 30 cm between the point at which the laser exits the tool 10 and the surface of the skin is sufficient to achieve the desired penetration and heating of the dermis to facilitate stimulation of the collagen and associated proteins retained therein.

Whilst the size of the spot of laser light applied to the skin is dependant upon the angle of incidence of the beam, the laser light is typically applied using a laser emission with a diameter of between 1-10 mm and preferably about 5 mm. In this regard, by controlled movement of the beam of laser light in a manner which correlates with the energy of the laser, the preferred elevation of skin temperature is achieved and careful movement of the spot of laser light can permit a substantial area of the skin to be treated in a dedicated treatment session.

The laser used in this treatment is a low energy CO₂ laser but other medically suitable substitutes could be used. Preferably, the laser light-utilised is a low energy CO₂ laser operated at a wavelength of about 10,600 nm operated at between about 0.5 and about 5 watts. However, other medically suitable lasers may be used to carry out the present invention. For example, any medically suitable laser operating at a wavelength in the range of about 400 to about 10,600 nm may be used, including an argon laser, an argon dye laser, a diode laser, a KTP laser, a copper vapour laser, a Yag laser, a ruby laser and a flashlamp-pumped dye laser. Further, an IPL (intense pulsed light) “laser” (though technically not a laser) may be used to achieve the results of the methods of the present invention. It is intended that the IPL (intense pulsed light) “laser” is included within the scope of lasers used in the present invention.

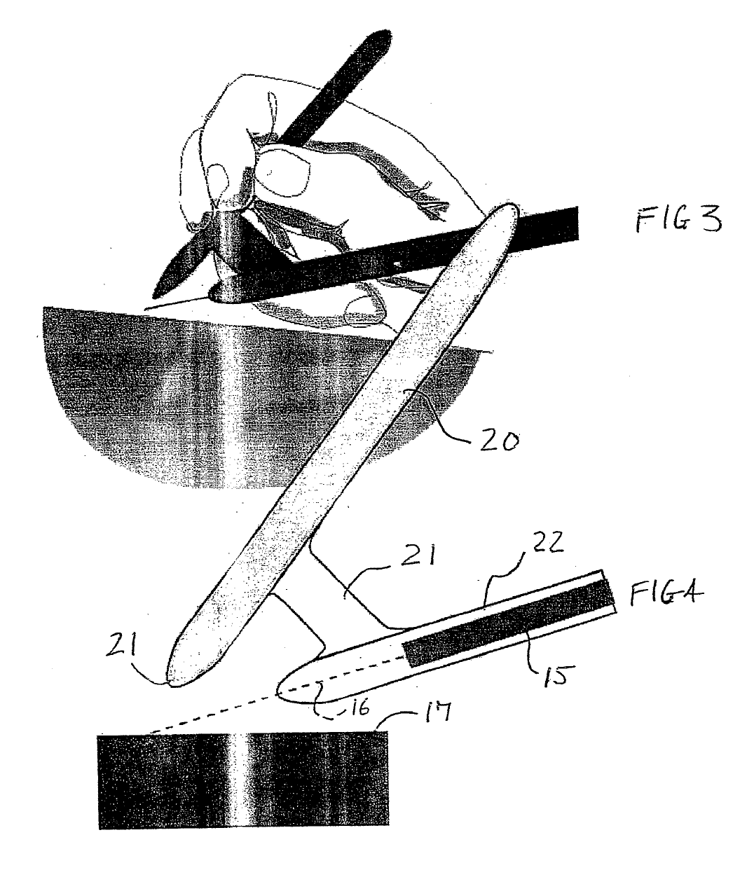

Referring now to FIGS. 3 and 4, a second embodiment of the device is provided. In this device, an elongate barrel 20 is adapted to be handheld as shown in FIG. 4 with its smooth tip 21 intended to be maintained spaced slightly from the surface of the patient's skin 17, but the smooth tip does in the alternative facilitate the device being applied to the skin and moved over the skin in contact. This device has a suspension strut 21 rigidly connected to the barrel 20 and supporting an angled cylindrical applicator 22 which accommodates the discharge unit 15 of a medical laser, whereby a beam of laser light 16 is discharged in a straight line through an opening in the tip of the barrel 22 to provide light incident on the skin at approximately 18°.

In instances where the treatment is being used to address conditions of irregular skin colouration, prior to applying the laser treatment, the affected area of the skin is firstly punctured with dry needles. Following the laser treatment, the area of laser treated skin may then be topically applied with comfrey and/or a moisturising cream to facilitate rapid healing.

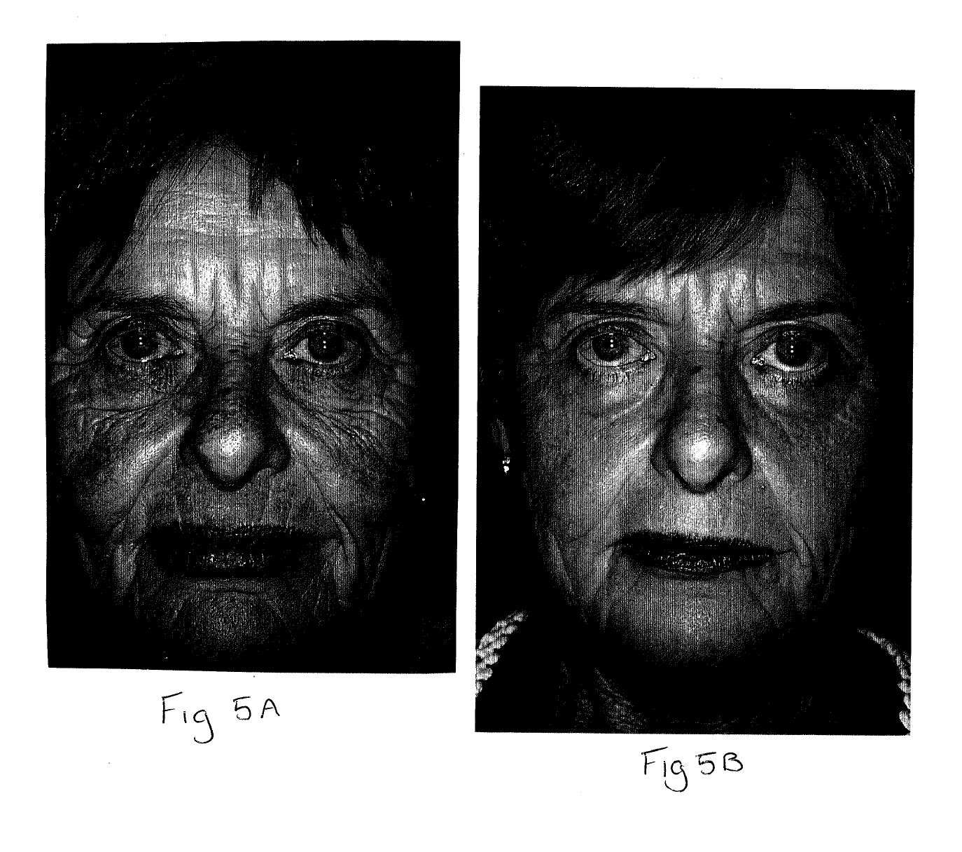

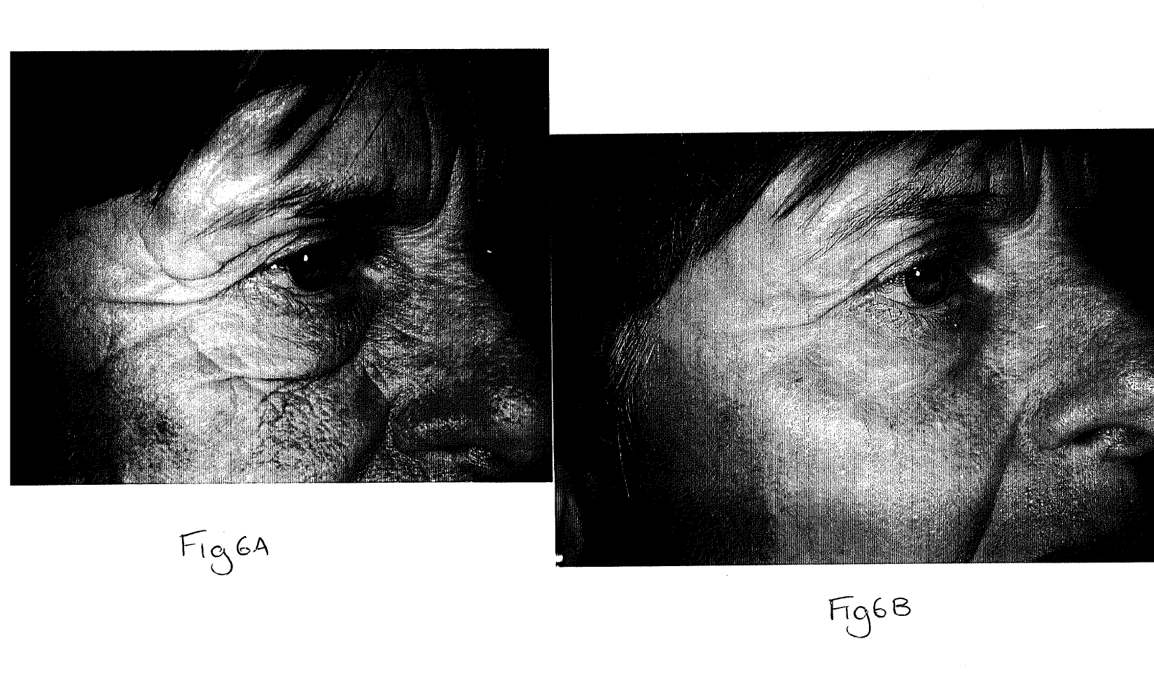

Referring now to FIGS. 5A and 5B and FIGS. 6A and 6B, before and after photographic depictions are shown of the treatment of the above described process. In each of these instances, an elderly patient received treatment for the somewhat delicate skin of the face and eye region and the effect after recovery was a significant tightening up of loose and aged skin. Whilst it may take a series of treatment procedures spaced several months apart to achieve the desired results, even a single treatment can be highly beneficial at remedying the problem.

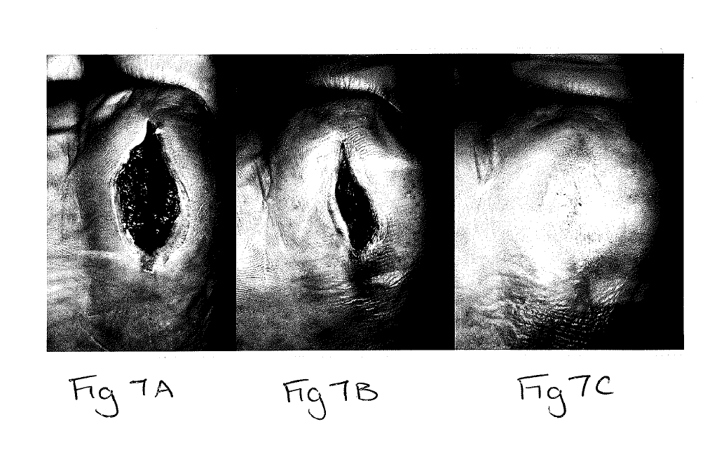

Referring to FIGS. 7A to 7C, photographic depictions are shown of the treatment of the above described process as applied to an open wound, such as an ulcer as shown. In this regard, the ulcer as shown in FIG. 7A had been present on the sole of the foot of a diabetic for 6 months prior to treatment. FIG. 7B shows the state of the ulcer six weeks following the first treatment, wherein healing of the wound is progressing. Finally, FIG. 7C shows the state of the ulcer two months after a second treatment, whereby the wound is completely closed and the skin growth rejuvenated to heal the wound.

It will be appreciated that this treatment may also be applied following an invasive surgical procedure to assist in fusing the opening of the surgical site and to prevent or reduce scarring at the surgical site. In this regard, the cut edges of the scar are melted and fused together to facilitate rapid healing at the surgical site and reduce pain associated with the surgery. Further, following the treatment the skin at the surgical site is inhibited from stretching after surgery, and the likelihood of scarring after surgery is reduced or eliminated.

The present invention can also be adapted to provide a treatment for stimulating hair growth or gum/mucosa regeneration in an area of skin or gum/mucosa where hair or gum/mucosa growth has receded or ceased or become sparse. This may be applied to hair loss arising from balding or stress or the recession of gum or mucosa growth arising from stress or disease. Similarly, the treatment can also be adapted to treat acne conditions of the skin.

In this application, the affected area of skin or gum/mucosa is applied with a laser light at an angle of incidence to the skin or gum/mucosa in the range of about 25° to 65°, most preferably about 45°, and from a distance between about 0.5 cm and about 30 cm from the skin or gum/mucosa. In this regard, the application of the laser light is conducted to confine the portion of skin under treatment to a temperature increase to a value between 0° C. to about 30° C. Typically, for treating acne in particular, there is a temperature increase in the portion of skin under treatment of about 5 to 30° C.

In another embodiment, it has been found that by increasing the distance of application of the laser, the present invention can be adapted to provide a method of laser-accelerated healing of a patient. This may have a significant and beneficial effect in treating many different conditions including burns, recent and chronic injuries, arthritis, stiff joints, skin conditions, active acne, psoriasis, dermatitis, warts, viral infections, allergies, asthma, internal bodily problems, assisting in pain management and wound healing.

In this application, the laser light is applied to the patient by waving the laser light at the patient from a distance between about 30 cm to 5 m from the patient. This method may be used on a patient in conjunction with any of the alternative embodiments described above and may be carried out either before or after carrying out any of the above described treatments.

This method includes using a wide zone of laser light applied to the skin at varying angles of incidence, the size of the spot depending on the angle of incidence. Generally, a laser emission with diameter 1 mm to 100 mm is used, but the size of the beam can vary outside this range and still function accordingly.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method of treating a skin condition or the like with a laser, comprising the steps of:
providing a laser energy source for generating a beam of laser energy;
applying the laser energy to the skin at an angle of incidence to the skin in the range of about 3° to 65°.

2. The method of treating a skin condition as recited in claim 1, wherein the laser energy is applied at an angle of incidence to the skin in the range of about 3° to 33°.

3. The method of treating a skin condition as recited in claim 1, wherein the laser energy is applied at an angle of incidence to the skin in the range of about 10° to 26°.

4. The method of treating a skin condition as recited in claim 1, wherein the laser energy is applied at an angle of incidence to the skin of about 18°.

5. The method of treating a skin condition as recited in claim 1, wherein the laser energy source is arranged at a distance of between 0.5 cm and 30 cm from the skin.

6. The method of treating a skin condition as recited in claim 1, wherein the laser energy is emitted at a distance of between 0.5 cm and 30 cm from the skin.

7. The method of treating a skin condition as recited in claim 1, further comprising the steps of:
prior to application of the laser energy, puncturing the skin with relatively dry needles, wherein,
the laser energy is applied at an angle of incidence to the skin in the range of about 3° to 33° and the laser energy source is in a non-contact arrangement with the skin.

8. The method of treating a skin condition as recited in claim 1, further comprising topically applying to the area of laser-treated skin comfrey or a moisturising cream or both to facilitate rapid healing.

9. The method of treating a skin condition as recited in claim 1, wherein the laser energy emission has a diameter of between about 2 mm to 10 mm in diameter.

10. The method of treating a skin condition as recited in claim 1, wherein the laser energy emission has a diameter of about 5 mm in diameter.

11. The method of treating a skin condition as recited in claim 1, wherein the dermis temperature of the treated skin is raised to a value in the range of 50° C. to 80° C.

12. The method of treating a skin condition as recited in claim 1, wherein the dermis temperature of the treated skin is raised to a value in the range of 50° C. to 65° C.

13. The method of treating a skin condition as recited in claim 1, wherein the method further comprises the step of:
providing a guide for receiving said laser energy source and for supporting said laser energy source above the skin, said guide having a laser energy discharge port through which said laser energy can pass from said laser energy source to the skin, said guide being positionable such that the device can be moved over and adjacent to the skin of the patient while maintaining an angle of incidence of the discharged laser energy to the skin.

14. The method of treating a skin condition as recited in claim 13, wherein the guide comprises a barrel having a tip adapted to translate over the surface of the skin, an opening near a tip, and a mirror located within the opening.

15. The method of treating a skin condition as recited in claim 13, wherein the guide comprises a barrel adapted to be handheld, a suspension strut extending from the barrel, and a generally elongated applicator selectively attached to the end of the suspension strut, the applicator receiving at least a portion of the laser energy source.

16. The method of treating a skin condition as recited in claim 1, wherein the skin condition is diminished hair growth, and wherein,
the laser energy is applied at an angle of incidence to the skin having diminished hair growth in the range of about 25° to 65°.

17. The method of treating a skin condition as recited in claim 1, wherein the skin condition is a surgical or open wound, and wherein,
the laser energy is applied to edges of the wound at an angle of incidence in the range of about 3° to 33° to fuse the edges together.

18. The method of treating a skin condition as recited in claim 1, wherein the skin condition is a disfigurement selected from the group consisting of scars, stretch marks, white scars, broken capillaries, and sun-damaged skin, and the laser energy is applied to the disfigured skin at an angle of incidence in the range of about 10° to 26°.

19. The method of treating a skin condition as recited in claim 1, wherein the laser energy source is a laser operated at between about 0.5 and about 5 watts.

20. The method of treating a skin condition as recited in claim 1, wherein the laser energy source is a CO₂ laser operated at a wavelength of about 10,600 nm.
